(19) United States
(12) Patent Application Publication
(10) Pub. No.: US 2020/0155467 A1
(43) Pub. Date: May 21, 2020

(54) METHOD AND SYSTEM FOR PRODUCTION OF SOFTGELS

(71) Applicant: TERRA LIFE SCIENCES INC., Calgary (CA)

(72) Inventors: ANIL JAIN, Calgary (CA); JERRY BECHTOLD, Calgary (CA)

(21) Appl. No.: 16/634,295

(22) PCT Filed: Jul. 27, 2018
(86) PCT No.: PCT/CA2018/050918

§ 371 Date: Jan. 27, 2020

Related U.S. Application Data
(60) Provisional application No. 62/538,367, filed on Jul. 28, 2017.

(51) Int. Cl. A61K 9/48

(52) U.S. Cl. CPC A61K 9/4833; A61K 9/4866

(57) ABSTRACT

A method and system for preparing softgels. A starch mixture is uniformly heated to an elevated temperature. The starch mixture includes plant starch, plasticizer and water, and does not include carrageenan. The starch mixture is heated to an elevated temperature under mixing for providing liquid starch, maintained at a maintenance temperature under mixing, and then cooled into a pair of starch sheets. Both sheets are provided to a die roller system with at least two pinch surfaces on each die, and sealed together between roller dies as individual softgels are cut by the dies. The die roller system is operated within a die temperature range and at a low die rpm range selected for providing the softgel.

### CROSS REFERENCE TO RELATED APPLICATIONS

This application claims the benefit of priority of U.S. Provisional Patent Application No. 62/538,367 filed Jul. 28, 2017, which is incorporated herein by reference in its entirety.

### FIELD

The present disclosure relates generally to production of softgels.

### BACKGROUND

Softgels are a popular delivery system for pharmaceuticals, natural health products, supplements, and other liquid or solid compounds. Softgels may be prepared from gelatin, which is derived from animals, plant-based starches, or any suitable material. Softgels manufactured from animal gelatin may be produced with an effective seal over a broader range of conditions compared with softgels manufactured from plant starch. However, gelatin softgels may suffer from odor or other failures when subjected to heat, humidity or other challenging conditions, to a greater extent than plant starch softgels. Some individuals will not use gelatin softgels for health, ethical, or religious reasons. To facilitate production of plant starch softgels with an effective seal, carrageenan is sometimes added to the softgel mixture as a thickening agent. Some individuals will not use carrageenan based on evidence that carrageenan may be associated with some negative effects on health.

### SUMMARY

Gelatin softgels suffer from drawbacks in terms of odour. In addition, gelatin softgels and plant starch softgels with carrageenan are avoided by some end-users for health, religion or ethical motivations. Production of plant starch softgels without carrageenan, and which have an effective seal, is challenging. It is, therefore, desirable to provide a softgel manufactured from plant starch without carrageenan, that does not leak medicinal ingredients and that forms an effective seal. It is an object of the present disclosure to obviate or mitigate at least one disadvantage of previous plant-based softgels.

Herein provided is a method and system for manufacturing softgels that includes heating a mixture of plant starch, plasticizer and water to an elevated mixing temperature under homogenous mixing conditions. No thickener or gelling agent, such as carrageenan, is required in the mixture. The plant starch, plasticizer and water may be provided in a weight ratio of approximately 10:3:12. The plant starch may include tapioca starch, potato starch, or any suitable plant starch, including starches activated for cross-linking. The plasticizer may include glycerin or any suitable plasticizer for softgel production. The homogenous mixing conditions may include blending the mixture with a combination of laminar and turbulent flow with forced axial flow. A combination of these types of mixing forces may facilitate mixing a highly viscous mixture and maintaining effective thermal heat conduction within the mixture. The mixing temperature may be between about 94 and about 98° C.

Once the mixture is heated to the mixing temperature, mixing is maintained for even thermal distribution throughout the mixture. Once the mixture is homogenized and at temperature, the mixture may be dropped to a lower maintenance temperature to maintain the mixture in a homogenous liquid state. The maintenance temperature may be between about 75° C. and about 85° C. The mixture may then be extended out into sheets of gelled plant starch and water. The sheets are rolled through a pair of rollers including dies for cutting out pieces of each sheet. The sheets are rolled at a low roller speed and the dies include at least two pinch surfaces. The roller speed may be between about 0.5 and about 2.0 revolutions per minute (“rpm”), and the dies may include three pinch surfaces.

In a first aspect, the present disclosure provides a method and system for preparing softgels. A starch mixture is uniformly heated to an elevated temperature. The starch mixture includes plant starch, plasticizer and water, and does not include carrageenan. The starch mixture is heated to an elevated temperature under mixing for providing liquid starch, maintained at a maintenance temperature under mixing, and then cooled into a pair of starch sheets. Both sheets are provided to a die roller system with at least two pinch surfaces on each die, and sealed together between roller dies as individual softgels are cut by the dies. The die roller system is operated within a die temperature range and at a low die rpm range selected for providing the softgel.

In a further aspect, the present disclosure provides a method of preparing a softgel comprising: preparing a liquid plant starch mixture free of carrageenan; providing the mixture to a softgel system, the softgel system comprising a pair of die rollers, the die rollers each comprising a plurality of dies, and the dies each comprising two or more pinch surfaces; providing a fill material to softgel system; and operating the softgel system at a die roller temperature and a die roller rpm value, resulting in the softgel, the softgel comprising the fill material encapsulated within the starch mixture.

In some embodiments, preparing the mixture comprises: heating a mixture of plant starch, plasticizer and water to a mixing temperature; mixing the mixture at the mixing temperature; and mixing the mixture at a maintenance temperature, the maintenance temperature being lower than the mixing temperature.

In some embodiments, the plant starch comprises tapioca starch or potato starch.

In some embodiments, the plant starch comprises a modified starch for facilitating cross-linking.

In some embodiments, the plasticizer comprises glycerin.

In some embodiments, the liquid plant starch mixture is free of any thickener.

In some embodiments, the mixture comprises the plant starch, the plasticizer and the water in a weight ratio of about 10:3:12.

In some embodiments, the mixing temperature is between 94° C. and 98° C.

In some embodiments, wherein the mixing temperature is 98° C.

In some embodiments, mixing the mixture at the mixing temperature comprises applying laminar and turbulent flow to the mixture for providing axial displacement to the mixture.

In some embodiments, wherein the maintenance temperature is between 70° C. and 90° C.

In some embodiments, the dies having two or more pinch surfaces comprise 3-pinch dies.

In some embodiments, the fill material comprises a pharmaceutical, a natural health product, a supplement.

In some embodiments, the die roller temperature is about 20° C.

In some embodiments, the die roller rpm value is about 1.2 rpm.

In a further aspect, herein provided is a method of preparing a softgel comprising: combining plant starch, plasticizer and water in a weight ratio of about 10:3:12; applying laminar and turbulent flow to the mixture for providing axial displacement to the mixture while heating the mixture to a mixing temperature of about 98° C.; maintaining the mixture at a maintenance temperature below the mixing temperature; providing the mixture to a 3-pinch softgel system at the maintenance temperature; providing a fill material to softgel system; and operating the softgel system at a die roller temperature of about 20° C. and a die roller rpm value of about 1.2 rpm, resulting in the softgel, the softgel comprising the fill material encapsulated within the starch mixture.

In some embodiments, the maintenance temperature is between 70° C. and 90° C.

In a further aspect, herein provided is a system for carrying out the methods described herein comprising a mixer for preparing the liquid starch; and a 3-pinch softgel roller die system.

In some embodiments, the mixer comprises an Ekato Unimix SRA 700 SO.

In some embodiments, the 3-pinch softgel system comprises a Sky softgel & Pack Co., Ltd SV-3000 system.

In a further aspect, herein provided is a vegan softgel capsule free of carrageenan prepared according to any of the methods described herein.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 is schematic of a system for producing softgels;

FIG. 2 is an elevation view of a die roller used in the system of FIG. 1;

FIG. 3 is a 3-pinch die from the die roller of FIG. 2; and

FIG. 4 is a cross section of a sidewall of a die from the die roller of FIG. 2.

### DETAILED DESCRIPTION

Softgels are one-piece, hermetically sealed soft shells for containing a fill material, which may be a liquid, a suspension, a semisolid or any flowable material. Softgels are generally formed by various processes including rotary die encapsulation. In rotary die encapsulation, two flexible sheets of gelatin, plant starch or other encapsulation material are synchronously guided over die rollers. The flexible sheets of encapsulation material are guided between the two dies rollers. The die rollers include recessed dies for determining the size and shape of the resultant softgel as the softgel shells are cut from the sheets. A displacement pump or other suitable flowable material delivery device provides the fill material into die cavities between the sheets. Negative pressure is applied to the dies, drawing solidifying sheets of gelatin into and against the die.

As the die rollers rotate, the die rollers pinch the sheets to seal the two sheets together, trapping the flowable material between the sheets. With further rotation, the dies cut into the sheets and pinch the sheets together, sealing a pair of die cut portions of each sheet together and enclosing the flowable material in a formed softgel. The die rollers may be cooled to facilitate cooling the two sheets of the starch and plasticizer mixture. As the dies cut into the sheets and pinch the resulting die cut portions together, an individual softgel is formed.

Current production challenges for softgels include mitigating leakage of the softgel and mitigating physical and chemical interactions between the shell and the fill components. Gelatin, which is derived from animals, provides good sealing under reasonable conditions, but is unpopular with some end-users for dietary, ethical, religious, sustainability or other reasons. Carrageenan is used as a thickener in plant starch mixtures to enhance sealing, but suffers in public perception as a result of publication of some evidence of toxicity following chronic exposure. Typical encapsulation approaches with gelatin or vegetable starch thickened by carrageenan will use a mixing temperature of about 70 to 80° C. and a flat die to manufacture softgels rapidly with die rollers rotating at 3 to 6 revolutions per minute (“rpm”). Multi-pinch dies are rarely used in softgel manufacture and are typically applied when the fill material is a greasy semi-solid such as lecithin. In applications with freely flowable oils and other payloads, single pinch dies are used with gelatin or vegetable starch thickened by carrageenan. The gelatin or carrageenan-thickened vegetable starch is mixed at a temperature of about 70 to 80° C. The methods and systems described herein apply an elevated mixing temperature, multiple pinch dies and low rpm values of the die rollers, to provide an effectively-sealed softgel prepared from plant starch without carrageenan.

Generally, the present disclosure provides a method and system for preparing vegan softgels without carrageenan. The method includes, and the system facilitates, preparing a liquid starch mixture and using the mixture to prepare the softgels with a roller die system. A mixture of plant starch, plasticizer and water is heated to an elevated mixing temperature under homogenous mixing conditions. The plant starch may include tapioca starch, potato starch, or any suitable plant starch, including starches activated for cross-linking. The plasticizer may include glycerin or any suitable plasticizer for softgel production. The mixture may include a weight ratio of approximately 10:3:12 plant starch, plasticizer and water. The starch may include tapioca starch, potato starch or any suitable starch mixture.

The mixing temperature may be at least 94° C. and may be between about 94 and about 98° C. The mixing conditions facilitate effective heat transfer and homogenization of a highly viscous mixture and may include blending the mixture with a combination of laminar and turbulent flow with forced axial flow. A combination of various mixing forces may facilitate mixing a highly viscous mixture and maintaining effective thermal heat conduction within the mixture. A Unimix SRA 700 SO mixing apparatus by Ekato may provide a suitable level of homogenization of a highly viscous mixture of plant starch and plasticizer in water. Mixing temperatures below about 94° C. appear to lack sufficient input energy to provide strong cross-linking and other reactions that provide a gelled material, while mixing temperatures above about 100° C. carry the risk of breaking down the starch polymers that will make up the soft gel upon cooling during encapsulation.

Once the mixture is heated to the mixing temperature, mixing is maintained for even thermal distribution throughout the mixture, and then may be dropped to a lower maintenance temperature to maintain the mixture in a homogenous liquid state. The maintenance temperature may be between about 75 and about 85° C.

Once homogenized, the mixture may be extended out across a large surface area to solidify into sheets upon cooling of gelled plant starch and water. The sheets are rolled through a pair of die rollers, which including dies for cutting out pieces of each sheet. The sheets are rolled at a low rpm under cooling the dies include at least two pinch surfaces. The roller speed may be between about 0.5 and about 2.0 rpm, such as about 1.2 rpm. The die rollers may be cooled to control the temperature of the gel sheet, facilitating cooling at a rate selected to provide a sealed softgel upon being cute by the multi-pinch die. With a die having at least two pinch surfaces, such as three pinch surfaces, the two sheets are engaged by the die to different extents one after the other.

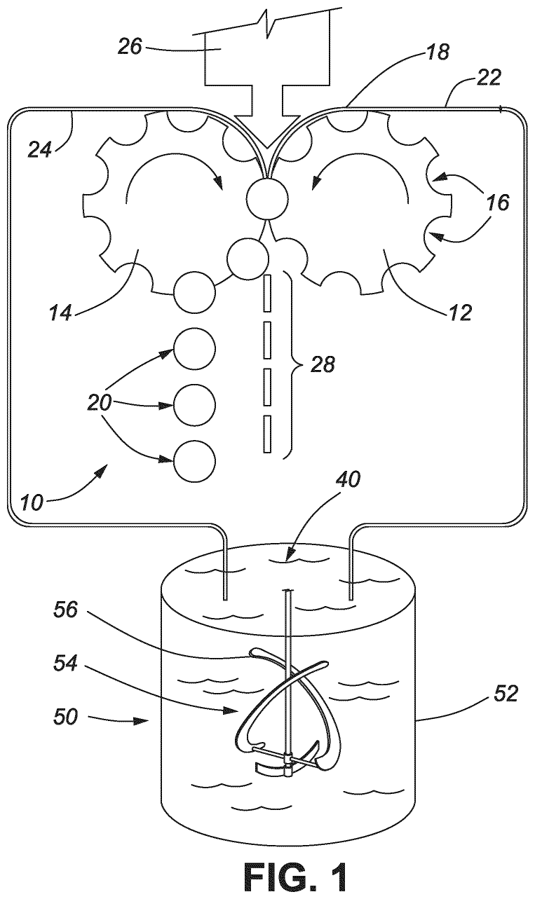

FIG. 1 shows a rotary die encapsulation system 10 for preparing softgels 20. The system 10 includes a first die roller 12 and a second die roller 14. Each of the first die roller 12 and the second die roller 14 defines multiple 3-pinch dies 16. In operation, a first starch sheet 22 rolls over the first die roller 12 and a second starch sheet 24 rolls over the second die roller 14. A fill material 26 is provided to a space 18 between the first die roller 12 and the second die roller 14. The fill material 26 is trapped in the space 18 between the first starch sheet 22 and the second starch sheet 24 as the first die roller 12 and the second die roller 14 rotate towards each other. The 3-pinch die 16 within which the fill material 26 is trapped cuts off the softgel 20, allowing remaining starch 28 to flow downward for recovery or disposal.

The first starch sheet 22 and the second starch sheet 24 are cooled from a starch mixture 40 that is prepared in a mixing and heating apparatus 50. The starch mixture includes plant starch, plasticizer and water. No carrageenan is necessary in the starch mixture 40 used in the methods and systems described herein. Other thickeners may be used, or modified starches may be applied to facilitate cross linking during formation of the softgel. The plant starch, plasticizer and water may be included in a weight ratio of about 10:3:12.

The heating and mixing apparatus 50 includes a body 52 and a mixer 54.

The body 52 may include fluid flow lines incorporated within it to allow heat exchange with the starch mixture 40 for heating the starch mixture 40 to an elevated mixing temperature and after mixing to be maintained at a maintenance temperature. Any other suitable heat exchange method may also be used as appropriate for a given heating and mixing apparatus 50. The mixer 54 is powered by rotation and includes mixing arms 56 for mixing the starch mixture 40. The mixing arms 56 may be curved for providing laminar and turbulent blending to the viscous starch mixture 40 inside the heating and mixing apparatus 50 upon rotation of the mixer 54. The laminar and turbulent mixing provides forced axial flow, resulting in homogenous mixing of the starch mixture 40 and effective transmission of heat throughout the starch mixture 40. The elevated mixing temperature may be about 98° C. and the maintenance temperature may be about 80° C.

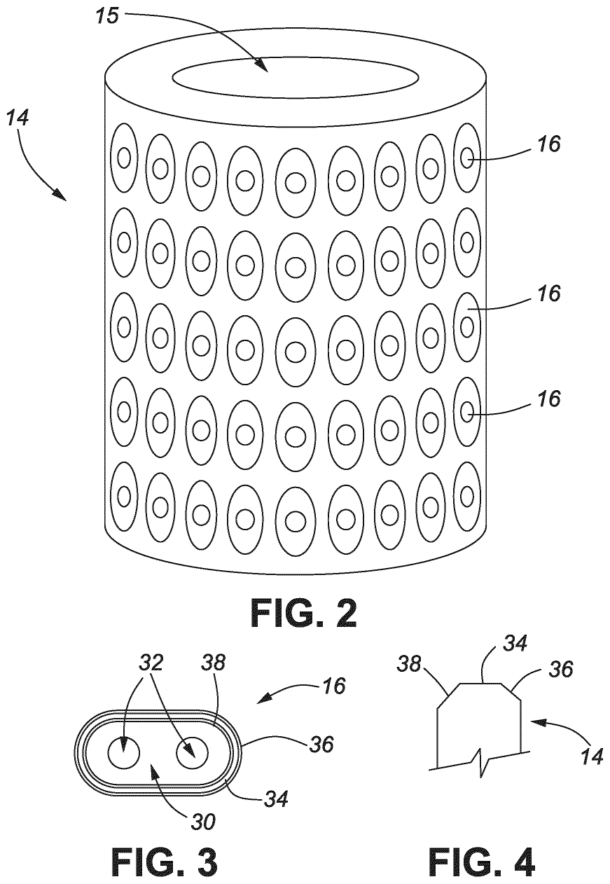

FIG. 2 shows the die roller 14 disconnected from the rotary die encapsulation system 10. A vacuum channel 15 is defined inside the die roller 14. The vacuum channel provides access to the dies 16. The die roller 12 would similarly include a vacuum channel (not shown).

FIG. 3 shows a die 16 as included on each of the first die roller 12 and the second die roller 14.

FIG. 4 shows a cross-section of a sidewall of the die 16. The die 16 includes a recessed portion 30 for receiving the first sheet 22 or the second sheet 24. A pair of suction access points are defined at the base of the recessed portion 30. The dies 16 shown in FIG. 2 are shown with a single vacuum access point.

The 3-pinch edge of the die 16 is apparent in FIGS. 3 and 4. A first pinch surface 36 is defined on an external face of the die 16. A second pinch surface 34 defines the 3-pinch edge adjacent to the first pinch surface 36. A third pinch surface 38 defines the 3-pinch edge adjacent to the second pinch surface 34. The second pinch surface 34 extends from the 3-pinch edge.

In operation, each surface of the 3-pinch edge becomes a pinch point to seal the first sheet 22 with the second sheet 24, encapsulating the fill material 28. During rotation, the first pinch surface 36 on each of two paired dies 16, from the first die roller 12 and the second die roller 14, is the first pinch point for the first sheet 22 and the second sheet 24, which are pinched together between the first die roller 12 and the second die roller 14. As rotation of the first die roller 12 and the second die roller 14 relative to each other proceeds, the respective dies 16 on each of the first die roller 12 and the second die roller 14 pinch the first sheet 22 and the second sheet 24 between the second pinch surface 34 of each of the dies 16. Finally, as rotation proceeds, the respective dies 16 on each of the first die roller 12 and the second die roller 14 pinch the first sheet 22 and the second sheet 24 between the third pinch surface 38 of each of the dies 16.

Rotation of the first die roller 12 relative to the second die roller 14 allows each of the first pinch surface 36, the second pinch surface 34, and the third pinch surface 38 to pinch the two sheets together, providing a seal in the resulting softgel 20. Having at least two pinch surfaces on the pinch edge provides greater opportunity for the first sheet 22 and the second sheet 24 to be pinched together. In addition, the rpm of the first die roller 12 and the second die roller 14 may be at a lower value that is often applied with gelatin sheets or vegetable starch sheets with carrageenan. The selected die roller rpm value depends on the system being used and other variables. The same system with animal gelatin would be operated at much greater die roller rpm value, such as about 4 rpm. In addition, a die roller temperature may be selected to maintain a suitable temperature for cooling the first sheet 22 and the second sheet 24, bonding the sheets together. The die roller temperature may be about 20° C.

On a Sky Softgel & Pack Co. Ltd SV-3000 encapsulation system with 3-pinch dies, a die roller rpm value of between about 1.0 and about 1.4 rpm, such as about 1.2 rpm, paired with a die roller temperature of between about 16° C. and about 24° C., such as about 20° C., was applied with success to a starch mixture of about 10:3:12 starch, gelatin and water that was mixed at 98° C. and maintained at 80° C. Die roller rpm values of between 0.5 and 2.0 rpm may also be applied.

The dies 16 are 3-pinch dies as shown in FIGS. 3 and 4. The dies 16 may be replaced with double-pinch dies or dies with a greater number of pinch surfaces, while maintaining the other aspects of the methods and systems described herein.

The methods and systems provided herein may be applied to creating a vegan softgel that performs well in terms of maintaining an effective seal and having desirable properties in terms of texture, firmness, elasticity and disintegration rate.

### Example I

In an example application of the methods and systems described herein, 40.820 kg of tapioca starch, 12.250 kg of glycerin (99% USP) and 46.930 kg of purified water USP were combined in an Ekato 600 kg gelatin receiver mixing vessel. The heat in the mixing vessel was set to 100° C. Mixing began at 25 rpm and ambient pressure conditions. When the gel mass reached 92° C., the mixing speed was increased to 35 rpm. When the gel mass reached 95° C., the set point was lowered to 98° C. and the mixing was maintained at 35 rpm for 30 minutes.

After the 30 minutes of mixing at 35 rpm ended, the mixing speed was lowered to 10 rpm and a negative pressure of −24 mmHg was applied to the mixing vessel and a vacuum valve was opened ¼ of the way, causing the starch mixture to rise. Once the starch mixture stopped rising, the valve was opened ½ way, causing the starch mixture to rise. Once the starch mixture stopped rising, mixing followed for 10 minutes. Once the 10 minutes ended, the mixer was turned off and allowed to mix under vacuum with no applied rotation for 1 minute.

After mixing, the vacuum was removed and air pressure was introduced. Once the air pressure increased above 4 on the reactor control screen, the starch mixture was transferred into liquid containers and maintained at 80° C. The starch mixture may be maintained at 70° C. to 90° C. for up to eight hours.

The starch mixture was used to provide two sheets of starch to a Sky Softgel & Pack Co. Ltd SV-3000 encapsulation system with 3-pinch dies. The die roller temperature was set to 20° C. A die roller rpm value was set to 1.2 rpm. At these settings, 500 mg of Life's Omega 055-0100 Algal Vegetable oil was added as a fill material to each resultant softgel, with a pumpstroke value of about 3.7 mm.

On a run of 7,000 softgels in the above method, 42 softgels were rejected. Of the 42 rejected softgels, 20 were leaking, 10 were twinned, and 12 were odd.

### Example II

The conditions of Example I were applied with 200 mg of fill material rather than 500 mg. On a run of 7,000 softgels in the above method, 30 softgels were rejected. Of the 30 rejected softgels, 10 were leaking, 5 were twinned, and 15 were odd.

### Example III

Multiple runs were operated with the following parameters:

TABLE 1
Parameters in Experiments of Example 3

| Starch (kg) | Glycerin (kg) | Water (kg) | Ratio |
|---|---|---|---|
| 100 | 30 | 116 | 10:3:12 |
| 126 | 38 | 144 | 10:3:12 |
| 75 | 23 | 87 | 10:3:12 |
| 171 | 23 | 225 | 15:2:20 |

In some cases, the starch was tapioca starch and in other cases the starch was potato starch. In the multiple runs with above weights of starch, glycerin and water, the number of successful runs generally matched

In the preceding description, for purposes of explanation, numerous details are set forth in order to provide a thorough understanding of the embodiments. However, it will be apparent to one skilled in the art that these specific details are not required. In other instances, well-known structures are shown in block diagram form in order not to obscure the understanding.

The above-described embodiments are intended to be examples only.

Alterations, modifications and variations can be effected to the particular embodiments by those of skill in the art. The scope of the claims should not be limited by the particular embodiments set forth herein, but should be construed in a manner consistent with the specification as a whole.

1. A method of preparing a softgel comprising:
preparing a liquid plant starch mixture free of carrageenan;
providing the mixture to a softgel system, the softgel system comprising a pair of die rollers, the die rollers each comprising a plurality of dies, and the dies each comprising two or more pinch surfaces;
providing a fill material to softgel system; and
operating the softgel system at a die roller temperature and a die roller rpm value, resulting in the softgel, the softgel comprising the fill material encapsulated within the starch mixture.

2. The method of claim 1 wherein preparing the mixture comprises:
heating a mixture of plant starch, plasticizer and water to a mixing temperature;
mixing the mixture at the mixing temperature; and
mixing the mixture at a maintenance temperature, the maintenance temperature being lower than the mixing temperature.

3. The method of claim 2 wherein the plant starch comprises tapioca starch or potato starch.

4. The method of claim 2 wherein the plant starch comprises a modified starch for facilitating cross-linking.

5. The method of claim 2 wherein the plasticizer comprises glycerin.

6. The method of claim 2 wherein the liquid plant starch mixture is free of any thickener.

7. The method of claim 2 wherein the mixture comprises the plant starch, the plasticizer and the water in a weight ratio of about 10:3:12.

8. The method of claim 2 wherein the mixing temperature is between 94° C. and 98° C.

9. The method of claim 8 wherein the mixing temperature is 98° C.

10. The method of claim 2 wherein mixing the mixture at the mixing temperature comprises applying laminar and turbulent flow to the mixture for providing axial displacement to the mixture.

11. The method of claim 2 wherein the maintenance temperature is between 70° C. and 90° C.

12. The method of claim 1 wherein the dies having two or more pinch surfaces comprise 3-pinch dies.

13. The method of claim 1 wherein the fill material comprises a pharmaceutical, a natural health product, a supplement.

14. The method of claim 1 wherein the die roller temperature is about 20° C.

15. The method of claim 1 wherein the die roller rpm value is about 1.2 rpm.

16. A method of preparing a softgel comprising:
combining plant starch, plasticizer and water in a weight ratio of about 10:3:12;
applying laminar and turbulent flow to the mixture for providing axial displacement to the mixture while heating the mixture to a mixing temperature of about 98° C.;
maintaining the mixture at a maintenance temperature below the mixing temperature;
providing the mixture to a 3-pinch softgel system at the maintenance temperature;
providing a fill material to softgel system; and
operating the softgel system at a die roller temperature of about 20° C. and a die roller rpm value of about 1.2 rpm, resulting in the softgel, the softgel comprising the fill material encapsulated within the starch mixture.

17. The method of claim 16 wherein the maintenance temperature is between 70° C. and 90° C.

18. A system for carrying out the method of claim 1 comprising
a mixer for preparing the liquid starch; and
a 3-pinch softgel roller die system.

19. The system of claim 18 wherein the mixer comprises an Ekato Unimix SRA 700 SO.

20. The system of claim 18 wherein the 3-pinch softgel system comprises a Sky softgel & Pack Co., Ltd SV-3000 system.

21. A vegan softgel capsule free of carrageenan prepared according to the method of claim 1.
